(19) United States
(12) Patent Application Publication
(10) Pub. No.: US 2012/0016185 A1
(43) Pub. Date: Jan. 19, 2012

(54) Vaginal Manipulator Tips and Related Systems and Methods

(76) Inventors: Charles Sherts, Westport, CT (US); Peter K. Arneson, Cheshire, CT (US)

(21) Appl. No.: 12/838,356

(22) Filed: Jul. 16, 2010

(51) Int. Cl. A61F 2/04

(52) U.S. Cl. 600/37

(57) ABSTRACT

This invention relates to vaginal manipulator tips and related systems and methods. In certain aspects of the invention, a vaginal manipulator tip assembly includes a body and an expandable member secured to the body. The tip assembly can be configured to be inserted into a vagina, and the body can be configured to receive a cervix.

### TECHNICAL FIELD

This invention relates to vaginal manipulator tips and related systems and methods.

### BACKGROUND

Vaginal prolapse is a condition that occurs when the network of muscles, ligaments, and skin in and around a woman's vagina weaken or break. As a result of the weakened muscles, ligaments, and skin, the vagina prolapses or falls out of its normal position. One type of vaginal prolapse referred to as a vaginal vault prolapse often occurs following a hysterectomy, which involves the removal of the uterus. In vaginal vault prolapse, the top of the vagina gradually falls toward the vaginal opening. This may cause the walls of the vagina to weaken as well. Eventually, the top of the vagina may protrude out of the body through the vaginal opening, effectively turning the vagina inside out.

Vaginal vault prolapses can be corrected with certain types of surgery, such as laparoscopic surgery. The surgical correction of this condition usually involves a technique called a vaginal vault suspension, in which the surgeon attaches the vagina to strong tissue in the pelvis or to a bone called the sacrum, which is located at the base of the spine.

### SUMMARY

In one aspect of the invention, a vaginal manipulator includes a vaginal manipulator shaft and a tip assembly secured to an end region of the vaginal manipulator shaft. The tip assembly includes a body and an expandable member secured to the body. The tip assembly is configured to be inserted into a vagina, and the body of the tip assembly is configured to receive a cervix.

In an additional aspect of the invention, a vaginal manipulator tip assembly includes a body and an expandable member secured to the body. The tip assembly is configured to be inserted into a vagina, and the body is configured to receive a cervix.

In a further aspect of the invention a method of correcting a vaginal prolapse includes inserting a vaginal manipulator tip into a vagina of a subject. The vaginal manipulator tip includes an expandable member secured to a body. The method also includes occluding a portion of the vagina by expanding the expandable member of the vaginal manipulator tip in the vagina, using the vaginal manipulator tip to move the vagina to a desired position, and securing the vagina to bone or tissue of the subject.

In another aspect of the invention, a vaginal manipulator includes a vaginal manipulator shaft including a tip hub that is pivotally connected to an elongate member, and a tip assembly configured to be secured to the tip hub of the vaginal manipulator shaft. The tip assembly includes a body and an expandable member secured to the body, and the tip assembly is configured to be inserted into a vagina.

Implementations can include one or more of the following features.

In certain implementations, the expandable member is configured to occlude the vagina when the tip assembly is inserted into the vagina and the expandable member is expanded.

In some implementations, the expandable member is further configured to create a substantially fluid-tight seal with the vagina when the tip assembly is inserted into the vagina and the expandable member is expanded.

In certain implementations, the expandable member is configured to inhibit the tip assembly from becoming disengaged from the vagina when the tip assembly is inserted into the vagina and the expandable member is expanded.

In some implementations, the expandable member is a balloon having a textured outer surface to increase friction between the outer surface of the balloon and the vagina.

In certain implementations, the expandable member is a balloon.

In some implementations, the expandable member is a mechanism that is configured to be radially expanded when an axial force is applied to the mechanism.

In certain implementations, the expandable member is a resilient cone-shaped member.

In some implementations, the expandable member is mounted between a proximal end and a distal end of the body of the tip assembly.

In certain implementations, the expandable member is mounted on the proximal end of the body of the tip assembly.

In some implementations, the tip assembly has a length that is at least 0.5 times a length of the vagina.

In certain implementations, the length of the tip assembly is greater than or equal to the length of the vagina.

In some implementations, the body of the tip assembly defines one or more recesses configured to receive at least a portion of the expandable member.

In certain implementations, the body of the tip assembly defines an annular recess configured to receive a resilient ring of the expandable member.

In some implementations, the body of the tip assembly defines an annular recess configured to receive substantially the entire expandable member when the expandable member is in an unexpanded configuration.

In certain implementations, the expandable member is releasably attached to the body.

In some implementations, the distal end region of the tip is configured to receive the cervix.

In certain implementations, the distal end region of the body includes at least one member that extends along a longitudinal axis of the body and has an inner surface. The cervix can be received adjacent the inner surface of the at least one member.

In some implementations, the inner surface of the at least one member is a curved surface.

In certain implementations, the distal end region of the body includes a plurality of members that extend circumferentially about the longitudinal axis of the body. The plurality of members define a recess between them that extends along the longitudinal axis of the body. This recess is configured to receive a cervix.

In some implementations, each of the plurality of members has a curved inner surface.

In certain implementations, the distal end region of the body defines a recess configured to receive the cervix.

In some implementations, the recess extends proximally from a distal end surface of the body.

In certain implementations, the recess has a depth of about 0.5 cm to about 5 cm (e.g., about 3.5 cm).

In some implementations, the recess has a diameter of about 0.5 cm to about 4.5 cm (e.g., about 3.5 cm).

In certain implementations, the manipulator shaft includes an elongate member with a proximal end portion and a distal end portion. The manipulator shaft also includes a tip hub connected to the elongate member and is configured to releasably receive and support the tip assembly.

In some implementations, the tip hub is pivotally connected to the distal end portion of the elongate member.

In certain implementations, the manipulator shaft includes a grip pivotally connected to the proximal end portion of the elongate member, and the grip is movable relative to the elongate member to control relative movements of the tip hub.

In some implementations, the elongate member has a curved configuration.

In certain implementations, the method of correcting vaginal prolapse further includes securing the vagina to the bone or tissue of the subject via a graft.

In some implementations, securing the vagina to the bone or tissue of the subject includes securing one end region of the graft to the vagina and an opposite end region of the graft to the bone or tissue.

In certain implementations, the one end region of the graft is sutured to the vagina and the opposite end region of the graft is sutured to the bone or tissue.

In some implementations, the method includes holding the vaginal manipulator tip adjacent a portion of the vagina being secured to the graft.

In certain implementations, the graft is a mesh member (e.g., a Y-shaped mesh member).

In some implementations, the method includes insufflating an abdominal cavity of the subject, wherein the expanded expandable member inhibits insufflation gas from escaping the subject via the vagina.

In certain implementations, the method includes the vaginal manipulator tip having at least one member that extends circumferentially about a longitudinal axis of the body. The method further includes rotating the vaginal manipulator tip within the vagina to position the at least one member adjacent a portion of the vagina being secured to the bone or tissue.

In some implementations, the method includes advancing the vaginal manipulator tip into the vagina until a cervix of the subject is received in a recess defined by a distal end portion of the vaginal manipulator tip.

Implementations can include one or more of the following advantages.

In certain implementations, the tip assembly is configured to be inserted into a vagina, and the expandable member is configured to occlude the vagina when the tip assembly is inserted into the vagina and the expandable member is expanded. By occluding the vagina, the tip assembly can inhibit insufflation gas from escaping the subject via the vagina during procedures in which the vagina is punctured, such as procedures to correct vaginal prolapse. Preventing insufflation gases from escaping via the patient's vagina can reduce or prevent the need to replenish the insufflation gas during the procedure. The expandable member can also advantageously prevent the tip assembly from slipping out of the vagina during a treatment.

In some implementations, the body of the tip assembly defines a recess configured to receive substantially the entire expandable member when the expandable member is in a non-expanded state. This arrangement can facilitate insertion of the tip assembly into a vagina. In particular, because the expandable member is flush with or recessed relative to the outer surface of the body of the tip assembly, the expandable member will typically apply little, if any, frictional force to the vaginal wall as the tip assembly is inserted into the vagina. The flush or recessed arrangement of the expandable member also improves the ease with which the vagina can be manipulated or repositioned within the patient by the tip assembly.

In certain implementations, the tip assembly is sized to extend along substantially the entire length of the vagina of a patient being treated (e.g., undergoing a vaginal prolapse correction procedure). This configuration can allow all of the sutures used to secure the vagina to a graft, tissue, or bone during the treatment to be placed without having to reposition the manipulator within the vagina. In addition, this configuration increases the ease with which the vagina can be manipulated by the tip assembly.

In some implementations, the distal end region of the body of the tip assembly is configured to receive a cervix of a patient being treated. This configuration can reduce interference caused by the cervix when repositioning or elevating the patient's vagina and can thus improve the surgeon's ability to elevate the vagina to its correct position within the patient. This configuration can also improve the surgeon's ability to secure a portion of the patient's vagina that is adjacent to (e.g., that surrounds) the cervix to a graft, tissue, or bone during the procedure.

In certain implementations, the manipulator shaft includes a pivotable tip hub to which the tip assembly can be connected. This gives the surgeon the ability to pivot the tip assembly during treatment. This provides the surgeon with the ability to manipulate the tip assembly within the vagina of a patient, which can facilitate insertion of the tip assembly into the vagina and can facilitate repositioning of the vagina. In addition, the pivotable tip assembly allows the surgeon to bias the vagina toward or away from the graft, tissue, or bone to which the vagina is being secured. This can improve the ability of the surgeon to suture or otherwise secure the vagina to the graft, tissue, or bone.

Other aspects, features, and advantages of the invention will be apparent from the description and drawings, and from the claims.

### DESCRIPTION OF DRAWINGS

FIG. 1 is a side view of a vaginal manipulator.

FIGS. 2A and 2B are side views of a tip assembly of the vaginal manipulator of FIG. 1 with a balloon of the tip assembly deflated and inflated, respectively.

FIGS. 3A-3C schematically illustrate a method of using the vaginal manipulator of FIG. 1 to perform a vaginal prolapse correction procedure, namely a sacral colpopexy procedure.

FIG. 4 is a side view of a vaginal manipulator tip assembly that includes a balloon positioned at the proximal end of a body of the tip assembly such that the balloon can be held securely between the body of the tip assembly and a tip hub of a vaginal manipulator shaft when the tip assembly is attached to the vaginal manipulator shaft.

FIG. 5 is a perspective view of a vaginal manipulator tip assembly having a body with a recessed portion that extends proximally from the distal end surface of the body and is configured to receive a cervix.

FIG. 6 is a cross-sectional view of the vaginal manipulator tip assembly of FIG. 5 positioned within the vagina of a patient such that the cervix of the patient is received within the recessed portion of the body of the tip assembly.

FIG. 7 is a perspective view of a vaginal manipulator tip assembly that includes opposed, elongate arcuate walls that form a central recess configured to receive a cervix.

FIG. 8 is a perspective view of a vaginal manipulator tip assembly that includes a single elongate arcuate wall that forms a recess configured to receive a cervix.

FIGS. 9A and 9B are side views of a vaginal manipulator tip assembly with an expandable mechanism in a non-expanded state and an expanded state, respectively.

FIG. 10 is a side view of a vaginal manipulator that includes a pivotable tip hub to which the tip assembly of FIGS. 2A and 2B is secured.

### DETAILED DESCRIPTION

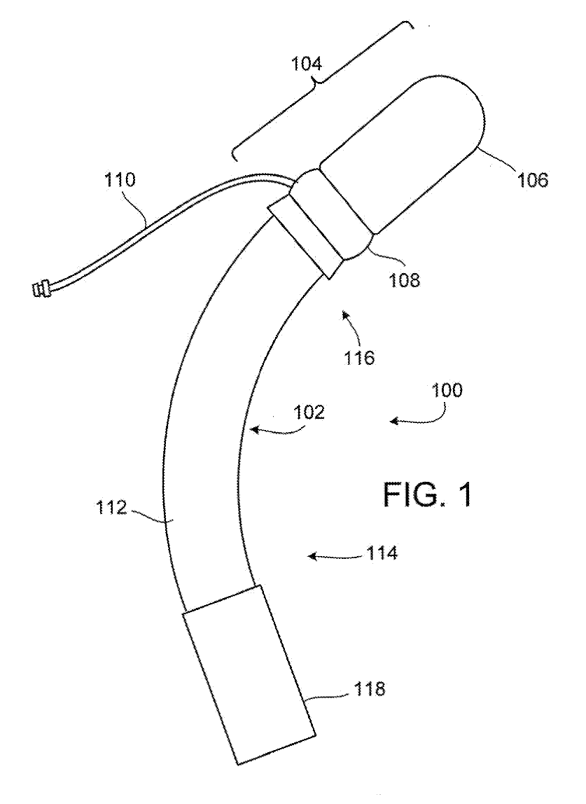

FIG. 1 illustrates a vaginal manipulator 100 that includes a vaginal manipulator shaft 102 and a tip assembly 104 releasably secured to a distal end region of the shaft 102. The tip assembly 104 includes a substantially cylindrical body 106 to which a balloon 108 is secured. Catheter tubing 110 is connected to the balloon 108 to allow the balloon 108 to be inflated and deflated as needed.

As will be described in greater detail below, the vaginal manipulator 100 can be used to reposition a prolapsed vagina during vaginal prolapse correction procedures, such as sacral colpopexy procedures. To reposition the prolapsed vagina, the user grasps the shaft 102 and inserts the tip assembly 104 assembly into the vagina. By moving the shaft 102, the tip assembly 104 and the vagina in which the tip assembly 104 is disposed can also be moved. With the tip assembly 104 disposed within the vagina, the balloon 108 can be inflated to create a substantially fluid-tight seal with the vaginal wall. This can help to prevent gases, such as insufflation gases, from escaping the patient via the patient's vagina. The inflated balloon 108 can also function to grip the vaginal wall to reduce (e.g., prevent) movement of the balloon 108 relative to the surrounding portion of the vagina. This can help to prevent the tip assembly 104 from becoming disengaged from or fall out of the vagina during a procedure.

The vaginal manipulator shaft 102 includes an elongate, arcuate shaft 112 having a proximal end portion 114 and a distal end portion 116. A handle 118 is coupled to the proximal end portion 114 of the arcuate shaft 112. The handle 118 can be integrally formed with the shaft 112 or can be formed as a separate piece and attached to the shaft 112. A suitable manipulator handle is commercially available from CooperSurgical (Trumbull, Conn.), under the name RUMI Arch™.

The arcuate shaft 112 and the handle 118 of the vaginal manipulator shaft 102 can be formed, e.g., molded and/or machined, from materials that are biocompatible and capable of withstanding heat-based medical device sterilization procedures (e.g., autoclave, steam autoclave, or dry heat oven) so that the vaginal manipulator shaft 102 can be sterilized and re-used after a treatment. Suitable materials that are capable of withstanding medical device sterilization procedures include metals, such as stainless steel and aluminum, and polymers, such as polyoxymethylene (POM) commonly known under the DuPont™ brand name Deirin®.

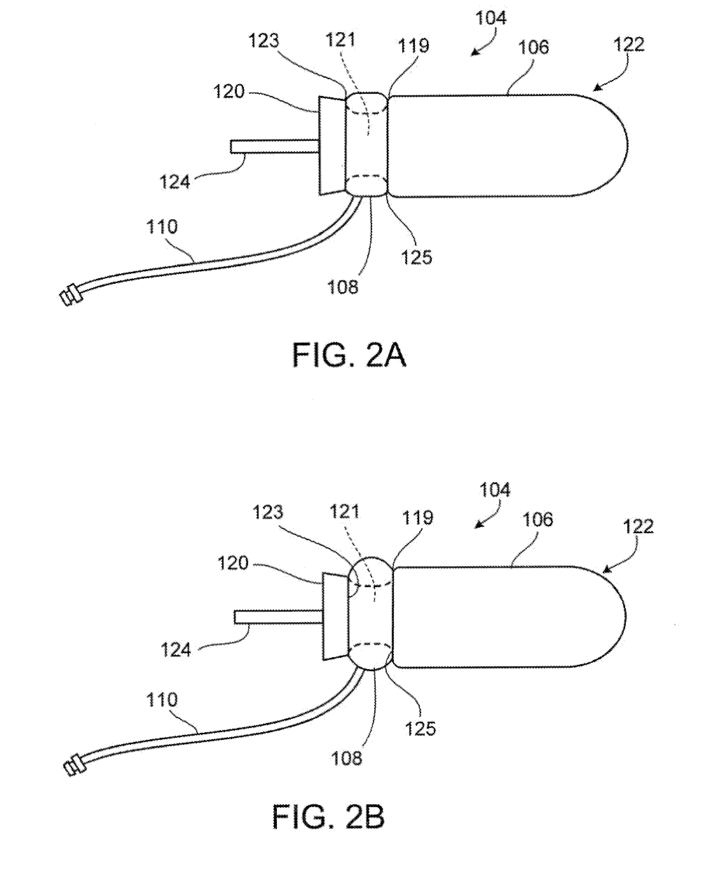

FIGS. 2A and 2B illustrate the tip assembly 104 of the vaginal manipulator 100 detached from the vaginal manipulator shaft 102 with the balloon 108 in a deflated state and an inflated state, respectively. Referring first to FIG. 2A, the body 106 of the tip assembly 104 has an annular recess 119 configured to receive substantially the entire ring-shaped or donut-shaped balloon 108 when the balloon 108 is deflated. This can facilitate insertion of the tip assembly 104 into a vagina by limiting the resistance or friction experienced by the balloon 108 contacting the vaginal walls during insertion. For similar reasons, this configuration can increase the ease with which the vagina can be manipulated or repositioned using the tip assembly 104.

The inner surface of the balloon 108 (i.e., the surface that forms the inner diameter of the donut-shaped balloon 108) is secured to a reduced diameter portion 121 of the body 106. In certain implementations, the balloon 108 is secured to the reduced diameter portion 121 of the body by a friction fit. In such implementations, the donut-shaped balloon 108 is formed of a resilient (e.g., elastic) material and has an inner diameter that, in a relaxed (i.e., unstretched) state, is less than the outer diameter of the reduced diameter portion 121 of the body 106. As a result, the balloon 108, when positioned around the reduced diameter portion 121 of the body 106 is stretched and secured to the body 106. The friction between the inner surface of the balloon 108 and the outer surface of the reduced diameter portion 121 of the body 106 in combination with end surfaces 123 and 125 of the body 106, which abut the proximal and distal ends of the balloon 108, limit movement of the balloon 108 relative to the body 106. As an alternative to or in addition to this stretch fit arrangement of the balloon 108, the inner surface of the balloon 108 can be bonded (e.g., thermally bonded or adhesively bonded using biocompatible adhesive) to the outer surface of the reduced diameter portion 121 of the body 106.

The inflation catheter or tube 110 is affixed to the balloon 108 and is in fluid communication with an interior of the balloon 108. The free end of the inflation tube 110 can be connected to a fluid source (e.g., a gas source or liquid source) and the fluid source can be operated to inflate the balloon 108. Any of various fluid sources, such as syringes and pumps, can be used to inflate the balloon 108.

The balloon 108 can be formed of any of various materials that permit the balloon 108 to form a substantially fluid-tight seal with the walls of a vagina when the balloon 108 is inserted into the vagina and inflated. Examples of suitable materials include high flexural modulus elastomers, such as thermo-plastic elastomers, silicone, polyethylene (PE), polyethylene terephthalate (PTE), and polyvinyl chloride. Other examples of suitable materials from which the balloon 108 can be formed include polyether-block-amide (e.g., Pebax®), nylon 12 (e.g., Vestamid®), and polyurethane elastomers (e.g., Tecothane®)

The body 106 of the tip assembly 104 has a length that is at least 0.5 times (e.g., at least about 0.75 times, at least about 1.0 times) the length of the vagina in which it is to be used. In some implementations, the body 106 has a length of about 5 cm to about 15 cm (e.g., about 8 cm to about 13 cm, about 13 cm). The body 106 also has a diameter that approximately matches the diameter of the vagina in which the tip assembly 104 is to be inserted. In certain implementations, the body 106 has a diameter of about 3 cm to about 6 cm (e.g., about 4 cm). The diameter of the reduced diameter portion 121 is typically about 0.1 cm to about 1.0 cm less than the diameter of adjacent larger diameter regions of the body 106. The body 106 has a rounded distal end region that generally conforms to the top region of a vagina. The rounded end region extends proximally about 1 cm to about 3 cm (e.g., about 1 cm) from the distal end of the tip assembly 104.

The body 106 of the tip assembly 104 can be formed, e.g., molded and/or machined, from polyoxymethylene. Alternatively or additionally, other biocompatible rigid thermoplastic or thermoset materials, such as polycarbonate (Lexan®) and acrylonitrile butadiene styrene (ABS), can be used to form the body 106 of the tip assembly 104. Typically the body 106 has a color that differs substantially from flesh (e.g., vaginal tissue) such that the body 106 can provide a distinct variance in color next to flesh and when flesh is stretched around the body 106. This can help a surgeon to distinguish the tip assembly 104 from the patient's flesh during surgical procedures.

As shown in FIG. 2B, when the balloon 108 is inflated, its diameter exceeds the maximum diameter of the body 106. In certain implementations, the diameter of the inflated balloon 108 is about 0.25 cm to about 4.0 cm greater than the maximum diameter of the body 106. The balloon 108 can, for example, have an outer diameter of about 3.5 cm to about 10 cm. Due to the size of the inflated balloon 108, when the tip assembly 104 is positioned within a vagina and the balloon 108 is inflated, the balloon 108 can create a fluid-tight seal with the walls of the patient's vagina.

The tip assembly 104 can be connected to the vaginal manipulator shaft 102 through an opening in the proximal end 120 of the body 106 of the tip assembly 104. A tip hub at the distal end 116 of the vaginal manipulator shaft 102 includes a pair of spaced apart flats which extend upwardly from a first surface of a base of the tip hub. The tip hub can be inserted into a counter-bore hole at the proximal end 120 of the tip assembly 104 and into a position in which the spaced apart flats engage a rectangular aperture in the proximal end 120 of the tip assembly 104. The tip hub also includes a hole for receiving a proximal end of a rod 124 designed to connect with the vaginal manipulator shaft 102. Further information regarding suitable mechanism for securing the tip assembly 104 to the vaginal manipulator shaft 102 can be found in U.S. Patent Application Publication No. 2010/0106163, which is incorporated by reference herein.

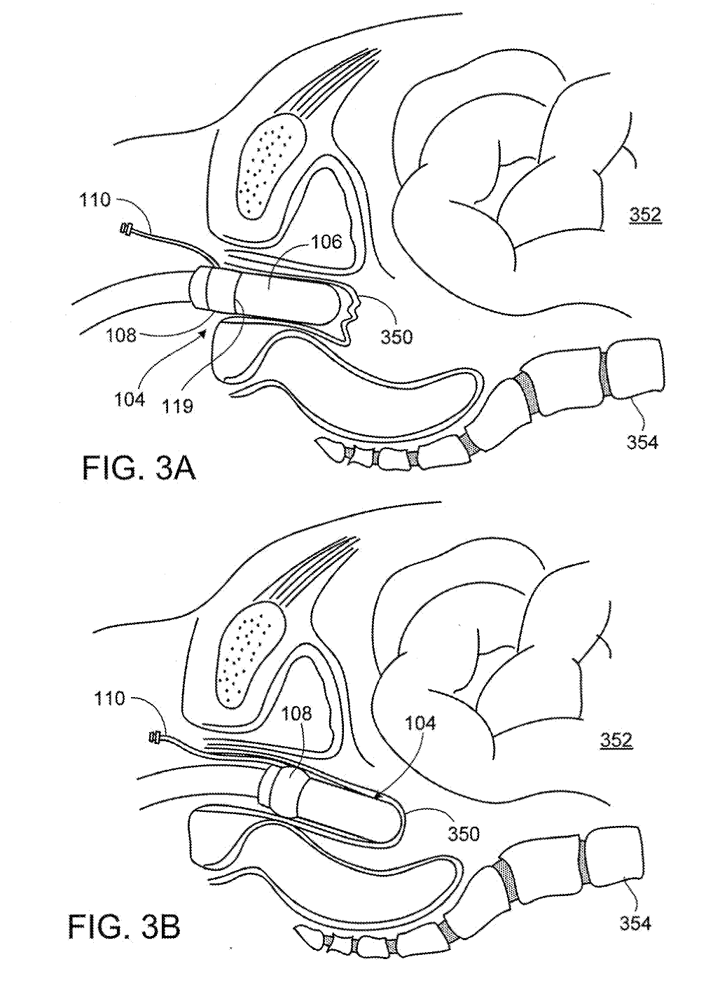

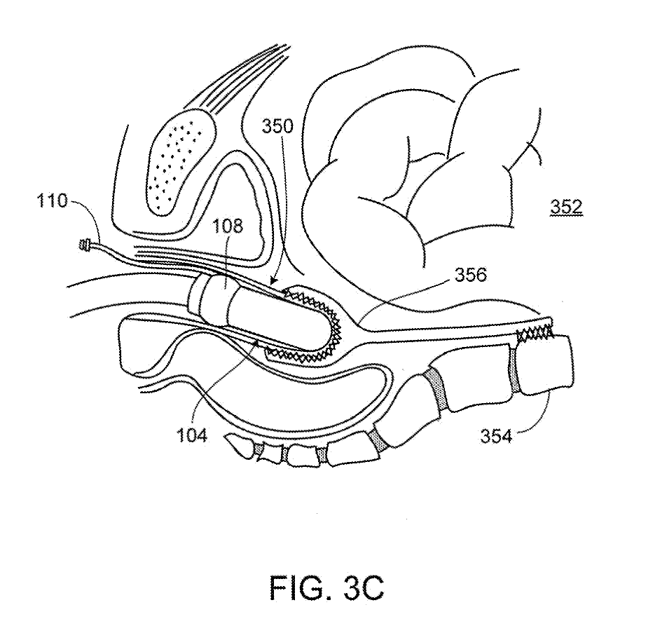

FIGS. 3A-3C schematically illustrate a method of using the vaginal manipulator 100 to reposition a patient's prolapsed vagina 350 during a vaginal prolapse correction procedure, namely a sacral colpopexy procedure. In this case, the patient previously underwent a hysterectomy procedure and, as a result, no longer has a uterus or cervix. Referring to FIG. 3A, the surgeon first grasps the shaft 102 of the vaginal manipulator 100 and inserts the tip assembly 104 into the prolapsed vagina 350. While inserting the tip assembly 104 into the vagina 350, the balloon 108 of the tip assembly 104 is in a deflated state such that the balloon 108 sits entirely or almost entirely within the recess 119 of the body 106 and does not substantially resist insertion of the tip assembly 104.

With the balloon 108 in the deflated state, the user continues to advance the tip assembly 104 into the vagina 350. The distal end region of the advancing tip assembly 104 eventually contacts the upper portion of the vagina 350, which has prolapsed into the vaginal cavity. As a result, advancement of the tip assembly 104 pushes the upper portion of the vagina 350 upward into the patient. The tip assembly 104 is advanced until the vagina 350 has been repositioned or raised to its proper location inside the patient, as shown in FIG. 3B.

Referring to FIG. 3C, after repositioning the vagina 350 as desired, a fluid source (e.g., a syringe or pump) is connected to the inflation tube 110 and operated to inflate the balloon 108. Prior to or after repositioning the vagina 350 and inflating the balloon 108, an abdominal cavity 352 of the patient is insufflated with insufflation gas (e.g., CO₂), and desired surgical instruments, including a laparoscope, are inserted into the abdominal cavity 352 of the patient. The insufflation of the abdominal cavity 352 improves accessibility to and visibility of the female pelvic organs for the surgeon.

The outer surface of the vagina 350 is then secured to a sacrum 354 of the patient via a Y-shaped graft 356. To secure the vagina 350 to the sacrum 354, the surgeon first sutures two branches of the Y-shaped graft 356, which can be made of synthetic material (e.g., polypropylene or polyester) or a natural material (e.g., fascia or dermis), to the anterior and posterior fascia of the vagina 350 using laparoscopic instruments. While suturing the Y-shaped graft 356 to the vagina 350, the tip assembly 104 of the vaginal manipulator 100 can help the surgeon to properly position the sutures within the vaginal tissue. The tip assembly 104 of the vaginal manipulator 100 can also be used in a manner similar to a thimble to provide a back-stop to aid in driving the needle and suture materials through the vaginal wall. Due to the length of the tip assembly 104, a portion of the tip assembly 104 typically sits adjacent the portion of the vagina 350 being sutured at all times. As a result, it is not typically necessary to move the tip assembly 104 relative to the vagina 350 during the suturing process.

The punctures created in the vagina 350 during this suturing process may cause insufflation gas to leak from the abdominal cavity 352 into the vaginal cavity of the patient. However, the inflated balloon 108 of the tip assembly 104 substantially prevents the insufflation gas from escaping the patient via the vagina 350 and thus helps to ensure that pneumoperitoneum is maintained. This can help to ensure that visibility within the abdominal cavity 352 of the patient remains adequate for the surgeon to complete the surgical procedure the procedure.

Still referring to FIG. 3C, once the two branches of the Y-shaped graft 356 have been secured to the vagina 350, the third branch of the Y-shaped graft 356 is sutured to the sacrum 354 to suspend the vagina 350 in the desired position. This is done using laparoscopic instruments that are positioned in the abdominal cavity 352 and thus have access to the outer wall of the vagina 350 and the sacrum 354. Once the suturing is completed, the insufflation gas is released from the abdominal cavity 352, and the balloon 108 is deflated. The vaginal manipulator 100 and the various laparoscopic surgical instruments are then removed from the patient.

While certain implementations have been described, other implementations are possible.

While FIGS. 3A-3C illustrate a sacral colpopexy procedure during which the vagina 350 of a patient is secured to the sacrum 354 of the patient, the vagina 350 can alternatively or additionally be attached via the graft 356 or a differently sized and/or shaped graft to other tissue or bone to correct the vaginal prolapse. For example, the vagina 350 can be secured to strong tissue in the pelvis of the patient. Alternatively or additionally, the vagina 350 can be secured to the uterosacral and/or scarospinous ligaments as well as other portions of the boney or ligamentous pelvic structure. During such procedures, the vaginal manipulator 100 is used in generally the same way to reposition the vagina and assist in the suturing process.

In addition, as an alternative to using a graft to secure the patient's vagina to bone or tissue, the vagina can be directly secured to the bone or tissue in certain cases.

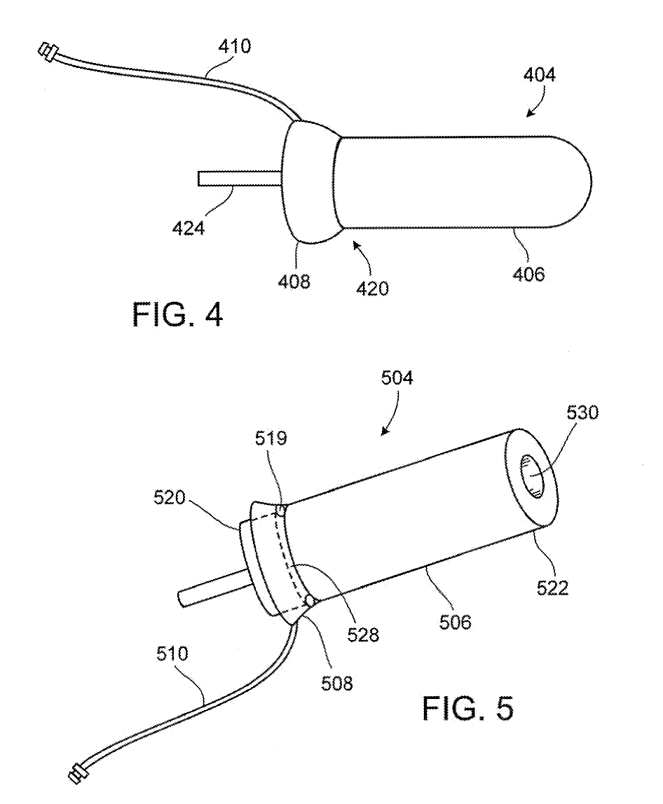

While the body 106 of the tip assembly 104 has been described as having the annular recess 119 in which the balloon 108 is disposed, alternative tip assemblies include bodies with no such annular recess. As shown in FIG. 4, for example, a tip assembly 404 includes a cylindrical body 406 having a substantially constant diameter along its length. A balloon 408 is secured to a proximal end 420 region of the body 406. In certain implementations, the inner surface of the balloon 408 is secured to a thin rod 424 in the proximal end 420 of the body 406 by friction fit. In such implementations, the donut-shaped balloon 408 is formed of a resilient material and has an inner diameter that, in a relaxed state, is less than the outer diameter of the rod 424. This rod 424 also fits into a hole in the vaginal manipulator shaft 102 that secures the tip assembly 404 to the vaginal manipulator shaft 102. As a result, the balloon 408 is further secured between a tip hub at the distal end region 116 of the vaginal manipulator shaft 102 and a rectangular aperture in the proximal end region 420 of the tip assembly 404. As an alternative or in addition to this stretch fit arrangement of the balloon 408, the inner surface of the balloon 408 can be bonded (e.g., thermally bonded or adhesively bonded using biocompatible adhesive) to the outer surface of the rod 424 and/or to the proximal end surface of the body 406. The tip assembly 404 can be connected to the vaginal manipulator shaft 102 and used in the same manner as the vaginal manipulator 100 described above.

While the vaginal manipulators above have been described as being used to reposition the vagina of a patient whose uterus was previously removed, vaginal manipulators can similarly be used to reposition vaginas in patients who still have their uterus or cervix. Such manipulators can include tip assemblies that are specially configured to accommodate the cervix of the patient during the procedure. FIG. 5 illustrates an example of one such vaginal manipulator tip assembly 504. As shown in FIG. 5, a distal end region 522 of a body 506 of the tip assembly 504 defines a recess 530 that extends proximally from a distal end surface of the body 506 and is configured to receive a cervix. The recess 530 has a diameter of about 0.5 cm to about 4.5 cm (e.g., about 3.5 cm) and/or a depth (measured along the longitudinal axis of the body) of about 0.5 cm to about 5.0 cm (e.g., about 3.5 cm). The body 506 of the tip assembly 504 can otherwise have dimensions similar to the body 106 of the tip assembly 104 described above and can be formed of any of the materials described above with respect to the body 106 of the tip assembly 104 described above.

A balloon 508 is secured to the body 506 of the tip assembly 504. A resilient ring 528 of the balloon 508 is secured within an annular recess 519 of the body 506. The resilient ring 528 and the annular recess 519 are configured so that the annular recess 519 receives the resilient ring 528 of the balloon 508 by friction fit. In particular, the inner diameter of the resilient ring 528, when the balloon 508 is in a relaxed (i.e., unstretched) state, is less than the outer diameter of the portion of the body 506 forming the annular recess 519. As a result, the resilient ring 528, when positioned around the annular recess 519, is stretched and the balloon 508 is secured to the body 506. The depth of the annular recess 519 can be further configured to be greater than the thickness of the resilient ring 528 of the balloon 508, further securing the resilient ring 528 in the annular recess 519 and restricting the balloon 508 from being able to move proximally or distally along the body 506 of the tip assembly 504. As an alternative to or in addition to this stretch fit arrangement of the balloon 508, the resilient ring 528 of the balloon 508 can be bonded (e.g., thermally bonded or adhesively bonded using biocompatible adhesive) to the portion of the body 506 forming the annular recess 519.

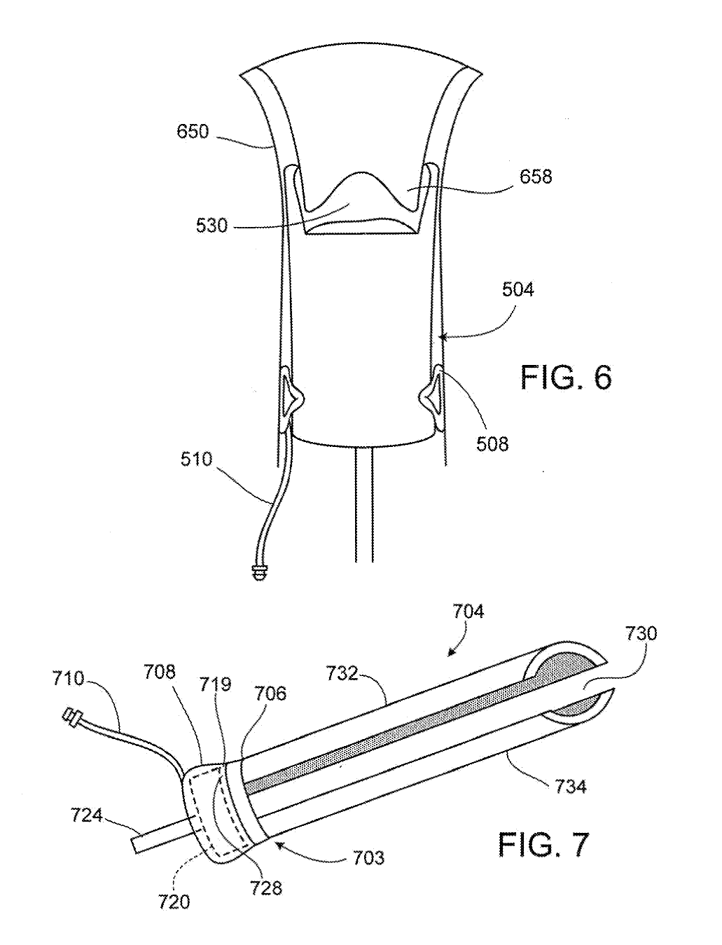

FIG. 6 is an anterior cross-sectional view of the tip assembly 504 with the tip assembly 504 fully inserted into a vagina 650 of a patient. As shown, a cervix 658 of the patient has been received in the recess 530 of the tip assembly 504. This allows the tip assembly 504 to be maneuvered in a way to restore the prolapsed vagina 650 to its original or desired position without the cervix 658 hindering the movement of the tip assembly 504. The length of the tip assembly 504 can help reduce or minimize the need for repositioning of the tip assembly 504 during the procedure. The balloon 508 can be inflated via an inflation tube 510. The balloon 508 is shown in its expanded configuration, occluding the vagina 650 to prevent insufflating gas from escaping the patient via the vagina 650.

While the bodies of the tip assemblies described above are generally cylindrical, tip assemblies having bodies of various other shapes can be used. Referring to FIG. 7, for example, a tip assembly 704 includes a body 706 having a proximal portion 703 and two opposed arcuate members 732, 734 that extend distally from the proximal portion 703. The arcuate members 732, 734 extend circumferentially about the longitudinal axis of the body 706 and have curved inner surfaces. A recess 730 sized to receive a cervix is formed between the two members 732, 734.

A balloon 708 is secured to the proximal portion 703 of the body 706. In particular, a resilient ring 728 of the balloon 708 is secured within an annular recess 719 formed in a proximal end region 120 of the body 706. The annular recess 719 is configured to receive the resilient ring 728 of the balloon 708 by friction fit. The balloon 708 can alternatively or additionally be secured to the body 706 using any of the various attachment techniques described above with respect to the balloons and bodies of other tip assemblies described herein.

The tip assembly 704 can be used in much the same way as the tip assembly 504 described above with respect to FIGS. 5 and 6. For example, the tip assembly 704 can be inserted into a prolapsed vagina, and the distal end of the tip assembly 704 can be used to push and reposition the prolapsed vagina into a desired location or orientation in the patient. As the distal end of the tip assembly 704 approaches the cervix of the patient, the recess 730 between the arcuate members 732, 734 receives the cervix. This allows a surgeon to fully advance the tip assembly 704 until the vagina is properly relocated. The surgeon can then, if necessary, rotate the tip assembly 704 such that the arcuate members 732, 734 sit adjacent the portion of the vagina being sutured. The opposing members 732, 734 allow for the surgeon to suture both anterior and posterior fascia of the outer vaginal wall once positioned. This can be advantageous in saving time and effort on the part of the surgeon. The balloon 708 can be inflated via an inflation tube 710 to occlude the vagina to prevent insufflation gas from escaping the patient via the patient's vagina.

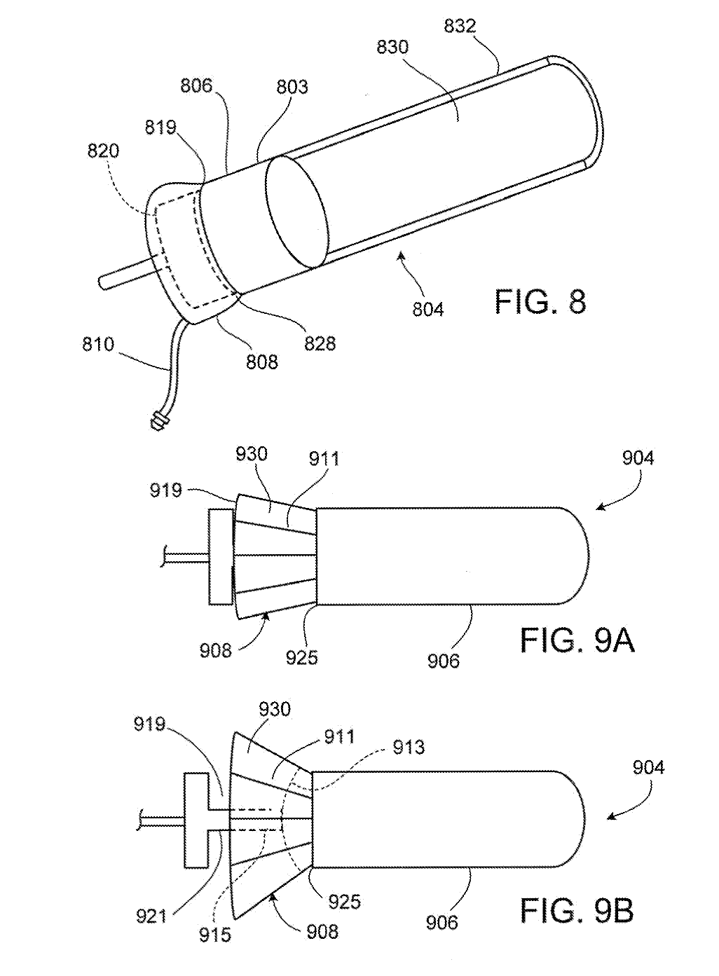

FIG. 8 illustrates a tip assembly 804 that is similar to the tip assembly 704 described above. However, the tip assembly 804 includes only a single arcuate member 832 that extends distally from a proximal portion 803 of a body 806 of the tip assembly 804. The member 832 extends circumferentially about a longitudinal axis of the body 806 and has a curved inner surface. During vaginal prolapse correction procedures in patients that still have their cervix, the cervix can be received in a recess 830 formed by the member 832. The cervix can be received in the recess 830 by maneuvering the member 832 between the cervix and the vaginal wall. This can help the operator manipulate the vagina even as it approaches the cervix and provide a brace against which sutures can be applied along the entirety of the vaginal wall. In addition, due to the relatively small volume occupied by the single member 832 of the body 806, the tip assembly 804 can be inserted into and guided through the patient's vagina relatively easily.

While the vaginal manipulator tip assemblies described above include balloons that can be inflated to create a substantially fluid-tight seal in a patient's vagina, other types of expandable members can be used for this purpose. In certain implementations, as shown in FIGS. 9A and 9B, for example, a vaginal manipulator tip assembly 904 includes an expandable mechanism 908 that is secured to a body 906 of the tip assembly 904. In an unexpanded state, as shown in FIG. 9A, an annular recess 919 formed in the body 906 receives substantially the entire expandable mechanism 908. The expandable mechanism 908 includes multiple first spokes 911 configured longitudinally and attached to the body 906 near a distal end 925 of the annular recess 919. A flexible biocompatible membrane 930 is attached to and surrounds the multiple spokes 911. Each first spoke 911 has a second corresponding spoke 913. The second spoke 913 is connected at its distal end to the first spoke 911 between the proximal and distal ends of the first spoke 911. The second spokes 913 are connected at their proximal ends to a ring 915 fitted around a reduced diameter portion 921 of the body 906. When expanded, as shown in FIG. 9B, the ring 915 is pushed distally up the reduced diameter portion 921 of the body 906. The ring 915 can, for example, be moved distally along the reduced diameter portion 921 of the body 906 by pushing a rigid wire that is attached to the ring 915 and extends proximally such that the wire is accessible to the surgeon during use. Pushing the ring 915 distally causes the distal ends of the second spokes 913 radially away from the body 906, forcing the proximal ends of the first spokes 911 radially outward. This expands the flexible cover of the expandable mechanism 908, creating a fluid-tight seal.

Any of various other types of expandable mechanisms can similarly be used with the tip assemblies described herein. For example, a mechanical iris, a coiled flat or wire spring, expanding bellows, or Chinese lantern-type supporting structures can be used for this purpose.

Other types of expandable members can alternatively or additionally be used with the tip assemblies described herein. For example, a flexible cone-shaped member can be secured around the body of a tip assembly. The outer diameter of a proximal end region of the cone-shaped member would be slightly larger than a vaginal canal of a patient while the outer diameter of the distal end region would be smaller than the vaginal canal. The cone-shaped member is formed of one or more flexible thermoplastic or thermoset materials, such as polyethylene, silicone, polyvinylchloride, and thermoplastic elastomers (TPEs). As the tip assembly is inserted into the vagina, the smaller distal end of the cone-shaped member readily passes through the vagina. As the larger proximal end region of the cone-shaped member reaches the vagina, it creates a substantially fluid-tight seal with the vaginal walls.

Multiple different sized vaginal manipulator tip assemblies of any of the various different types described above can be provided in a kit. The kit can also include the vaginal manipulator shaft. Prior to treatment, the surgeon can select the tip assembly that most closely matches the size of the vagina to be treated and can then use that tip assembly during the vaginal prolapse correction procedure. Selecting a tip assembly that closely matches the patient's vagina size improves the ability of the surgeon to manipulate or reposition the patient's vagina after the tip assembly has been inserted into the vagina. Selecting a tip assembly having a length that is substantially equal to the length of the patient's vagina also helps to ensure that the tip assembly will be positioned adjacent each of the regions of the vagina to be sutured during the procedure and can thus facilitate the suturing process.

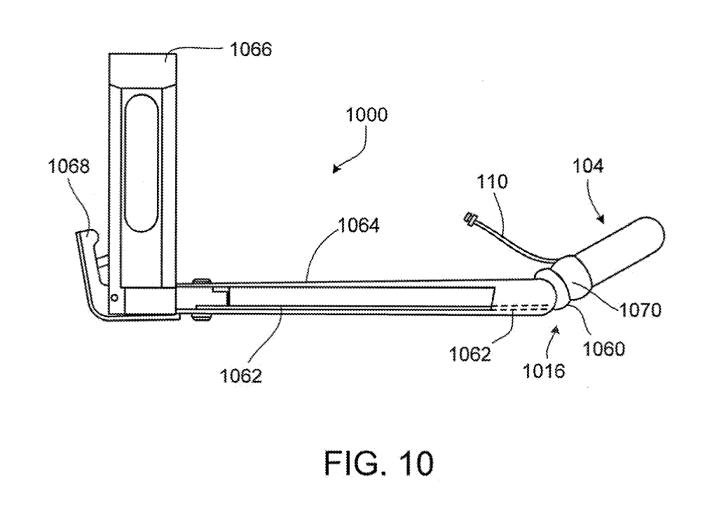

While the tip assemblies above have been described as being attached to the arcuate vaginal manipulator shaft 102, the tip assemblies can alternatively be used with other types of vaginal manipulator shafts. Referring to FIG. 10, for example, the tip assembly 104 described above is shown attached to an articulate shaft 1000. The shaft 1000 has a pivotable joint 1060 in its distal end region 1016. The pivotable distal end region 1016 can help in positioning the tip assembly 104 within the vaginal canal, and also in manipulating the vagina with the tip assembly 104. The tip assembly 104 is pivotally controlled by a connecting structure 1062 that operatively couples a tip hub 1070 to a grip 1066 such that movement of the grip 1066 relative to a frame 1064 causes a corresponding movement of the tip hub 1070 relative to the frame 1064. The connecting structure 1062 can, for example, be a wire. A locking mechanism 1068 is operable to fix the grip 1066 against movement relative to the frame 1064.

While the vaginal manipulators above have been described as being used during laparoscopic surgical procedures to correct vaginal prolapse, the vaginal manipulators can be used in a similar manner during open surgical procedures (e.g., laparotomy procedures) to correct vaginal prolapse.

While the vaginal prolapse correction procedures described above involve the surgeon grasping and maneuvering the shaft of the vaginal manipulator in order to reposition the patient's vagina, an automated device, such as a robotic arm, can alternatively be used to grasp and maneuver the vaginal manipulator shaft in order to reposition the patient's vagina.

While the vaginal manipulators above have been described as being used during vaginal prolapse correction procedures, the vaginal manipulators can be used for other types of procedures, such as laparoscopic procedures, including hysterectomy, removal of the adnexa, treatment of endometriosis, and other gynecologic diagnostic and therapeutic procedures. These manipulators can also be used for gynecologic procedures done at laparotomy.

Other implementations are within the scope of the following claims.

What is claimed is:

1. A vaginal manipulator, comprising:
a vaginal manipulator shaft;
a tip assembly secured to an end region of the vaginal manipulator shaft, the tip assembly comprising a body and an expandable member secured to the body,
wherein the tip assembly is configured to be inserted into a vagina, and the body of the tip assembly is configured to receive a cervix.

2. The vaginal manipulator of claim 1, wherein the expandable member is configured to occlude the vagina when the tip assembly is inserted into the vagina and the expandable member is expanded.

3. The vaginal manipulator of claim 2, wherein the expandable member is configured to create a substantially fluid-tight seal with the vagina when the tip assembly is inserted into the vagina and the expandable member is expanded.

4. The vaginal manipulator of claim 1, wherein the expandable member is a balloon.

5. The vaginal manipulator of claim 1, the expandable member is a mechanism that is configured to be radially expanded when an axial force is applied to the mechanism.

6. The vaginal manipulator of claim 1, wherein the expandable member is a resilient cone-shaped member.

7. The vaginal manipulator of claim 1, wherein the expandable member is mounted between a proximal end and a distal end of the body of the tip assembly.

8. The vaginal manipulator of claim 1, wherein the expandable member is mounted on the proximal end of the body of the tip assembly.

9. The vaginal manipulator of claim 1, wherein the tip assembly has a length that is at least 0.5 times a length of the vagina.

10. The vaginal manipulator of claim 9, wherein the length of the tip assembly is greater than or equal to the length of the vagina.

11. The vaginal manipulator of claim 1, wherein the body defines one or more recesses configured to receive at least a portion of the expandable member.

12. The vaginal manipulator of claim 11, wherein the body defines an annular recess configured to receive a resilient ring of the expandable member.

13. The vaginal manipulator of claim 11, wherein the body defines an annular recess configured to receive substantially the entire expandable member when the expandable member is in an unexpanded configuration.

14. The vaginal manipulator of claim 1, wherein the expandable member is releasably attached to the body.

15. The vaginal manipulator of claim 1, wherein a distal end region of the body is configured to receive the cervix.

16. The vaginal manipulator of claim 15, wherein the distal end region of the body comprises at least one member that extends along a longitudinal axis of the body and has an inner surface, and the cervix can be received adjacent the inner surface of the at least one member.

17. The vaginal manipulator of claim 16, wherein the inner surface of the at least one member is a curved surface.

18. The vaginal manipulator of claim 15, wherein the distal end region of the body comprises a plurality of members that extend circumferentially about the longitudinal axis of the body, and the plurality of members define a recess therebetween that extends along the longitudinal axis of the body and is configured to receive the cervix.

19. The vaginal manipulator of claim 18, wherein each of the members has a curved inner surface.

20. The vaginal manipulator of claim 15, wherein the distal end region of the body defines a recess configured to receive the cervix.

21. The vaginal manipulator of claim 20, wherein the recess extends proximally from a distal end surface of the body.

22. The vaginal manipulator of claim 1, wherein the manipulator shaft comprises an elongate member having a proximal end portion and a distal end portion, and a tip hub connected to the elongate member and configured to releasably receive and support the tip assembly.

23. The vaginal manipulator of claim 22, wherein the tip hub is pivotally connected to the distal end portion of the elongate member.

24. The vaginal manipulator of claim 23, wherein the manipulator shaft further comprises a grip pivotally connected to the proximal end portion of the elongate member, and the grip is movable relative to the elongate member to control relative movements of the tip hub.

25. The vaginal manipulator of claim 22, wherein the elongate member has a curved configuration.

26. A vaginal manipulator tip assembly, comprising:
a body; and
an expandable member secured to the body,
wherein the tip assembly is configured to be inserted into a vagina, and the body is configured to receive a cervix.

27. The vaginal manipulator tip assembly of claim 26, wherein the tip assembly has a length that is at least 0.5 times a length of the vagina.

28. The vaginal manipulator tip assembly of claim 27, wherein the length of the tip assembly is greater than or equal to the length of the vagina.

29. The vaginal manipulator tip assembly of claim 26, wherein a distal end region of the body is configured to receive the cervix.

30. The vaginal manipulator tip assembly of claim 29, wherein the distal end region of the body comprises at least one member that extends along a longitudinal axis of the body and has an inner surface, and the cervix can be received adjacent the inner surface of the at least one member.

31. The vaginal manipulator tip assembly of claim 29, wherein the distal end region of the body comprises a plurality of members that extend circumferentially about the longitudinal axis of the body, and the plurality of members define a recess therebetween that extends along the longitudinal axis of the body and is configured to receive the cervix.

32. The vaginal manipulator tip assembly of claim 26, wherein the distal end region of the body defines a recess configured to receive the cervix.

33. The vaginal manipulator tip assembly of claim 32, wherein the recess extends proximally from a distal end surface of the body.

34. A method of correcting vaginal prolapse, the method comprising:
inserting a vaginal manipulator tip into a vagina of a subject, the vaginal manipulator tip comprising an expandable member secured to a body;
occluding a portion of the vagina by expanding the expandable member of the vaginal manipulator tip in the vagina;
moving the vagina, using the vaginal manipulator tip, to a desired position; and
securing the vagina to bone or tissue of the subject.

35. The method of claim 34, wherein the expandable member is a balloon.

36. The method of claim 35, wherein the vagina is secured to the bone or tissue of the subject via a graft.

37. The method of claim 36, wherein securing the vagina to the bone or tissue of the subject comprises securing one end region of the graft to the vagina and an opposite end region of the graft to the bone or tissue.

38. The method of claim 37, wherein the one end region of the graft is sutured to the vagina and the opposite end region of the graft is sutured to the bone or tissue.

39. The method of claim 37, further comprising holding the vaginal manipulator tip adjacent a portion of the vagina being secured to the graft.

40. The method of claim 36, wherein the graft is a Y-shaped mesh member.

41. The method of claim 34, further comprising insufflating an abdominal cavity of the subject, wherein the expanded expandable member inhibits insufflation gas from escaping the subject via the vagina.

42. The method of claim 34, wherein the body of the vaginal manipulator tip comprises at least one member that extends circumferentially about a longitudinal axis of the body.

43. The method of claim 42, further comprising rotating the vaginal manipulator tip within the vagina to position the at least one member adjacent a portion of the vagina being secured to the bone or tissue.

44. The method of claim 34, wherein further comprising advancing the vaginal manipulator tip into the vagina until a cervix of the subject is received in a recess defined by a distal end portion of the vaginal manipulator tip.

45. A vaginal manipulator, comprising:
a vaginal manipulator shaft comprising a tip hub that is pivotally connected to an elongate member;
a tip assembly configured to be secured to the tip hub of the vaginal manipulator shaft, the tip assembly comprising a body and an expandable member secured to the body,
wherein the tip assembly is configured to be inserted into a vagina.

46. The vaginal manipulator of claim 45, wherein the manipulator shaft further comprises a grip pivotally connected to a proximal end portion of the elongate member, and the grip is movable relative to the elongate member to control relative movements of the tip hub.
